(19) United States
(12) Patent Application Publication
(10) Pub. No.: US 2012/0053117 A1
(43) Pub. Date: Mar. 1, 2012

(54) PHARMACEUTICAL COMPOSITION, DRUG SCREENING METHOD AND METHOD FOR TREATING MALARIA

(75) Inventors: Célia Regina da Silva Garcia, Carapicuiba (BR); Bettina Malnic, Madalena (BR); Luciana Madeira da Silva, Pocos de Caldas (BR); Pedro Alexandre Favoretto Galante, Liberdade (BR)

(73) Assignees: Fundacao De Amparo A Pesquisa Do Estado De Sao Paulo- FAPESP, Sao Paulo (BR); Universidade de Sao Paulo-USP, Sao Paulo (BR)

(21) Appl. No.: 13/119,707

(22) PCT Filed: Oct. 2, 2009
(86) PCT No.: PCT/BR2009/000331

§ 371 Date: Apr. 1, 2011

(30) Foreign Application Priority Data
Oct. 3, 2008 (BR) PI0804314-0

(51) Int. Cl. A61K 38/02; A61K 31/20; G01N 33/566; A61P 33/06

(52) U.S. Cl. 514/4.4; 514/558; 435/7.21

(57) ABSTRACT

The present invention relates to the use of serpentine receptors existent in parasites of genus Plasmodium for treating malaria.

### FIELD OF APPLICATION

The present invention is applied to the pharmaceutical area for the production of antimalaricals.

### PRIOR ART

Malaria is the worst human parasitic disease, which etiological agent is the protozoan of Plasmodium genus. Every year around 500 Million people are infected, causing the death of almost 2-3 Million of African children per year. In Brazil, the number of cases in Legal Amazonia has showed an increase of 25% since 2002, with around 460 thousand cases in the year 2004, which has also been followed by an increase of 27% in the malaria number of cases proportion caused by P. falciparum, the species responsible for the most lethal form of such disease (Garcia C R S, Azevedo M F, Wunderlich G, Budu A, Young J and Bannister L. G (2008) Plasmodium in the Post Genome Era: New insights into the molecular cell biology of the malaria parasites. International Review of Molecular and Cell Biology 266: 85-156).

Despite of countless efforts towards the malaria control, the number of cases continues to increase due to arising of parasites resistant to most available antimalaricals, as well as insecticides-resistant mosquitoes, which makes necessary to develop alternative strategies to eradicate such disease. In this sense, one of the huge obstacles is the complexity of malaria parasites and their interactions with the human host and vector-insect.

### Life-Cycle of Malaria Parasite: Parasite-Host Interactions

Asexual cycle of P. falciparum occurs in human host, and the infection begins with the bite of female anopheles mosquito, which injects sporozoites with saliva. Recently, it was proven that firstly injected sporozoites cross through dermis and only a few of them go into the capillary vessels, while others go into lymph vessels and originate exoerythrocytic forms unknown until then, which may have an important influence on host immunological system (Amino R, Thiberge S, Martin B, Celli S, Shorte S, Frischknecht F & Menard R (2006) Quantitative imaging of Plasmodium transmission from mosquito to mammal. Nat Med 12: 220-224). Once in the bloodstream, sporozoites invade hepatocytes and develop themselves in exoerythrocytic forms, which rupture the cells releasing merozoites in the blood (Mota M M, Pradel G, Vanderberg J P, Hafalla J C R, Frevert U, Nussenzweig R S, Nussenzweig V & Rodriguez A (2001) Migration of Plasmodium sporozoites through cells before infection). Merozoites invade erythrocytes and develop themselves inside the parasitophorous vacuoles, suffering several biochemical and morphological changes that may basically be identified by three stages known as ring, trophozoite and schizont. Erythrocyte rupture releases merozoites allowing continuity of intraerythrocytic cycle (Bannister L H, Hopkins J M, Fowler R E, Krishna S & Mitchell G H (2000) A brief illustrated guide to the ultrastucture of Plasmodium falciparum asexual blood stages. Parasitol Today 16: 427-433).

Some parasites in bloodstream develop into gametocytes, which are the infective form for the vector mosquito, where the sexual cycle occurs. In the mosquito bowel occurs the maturation of gametocytes, a process known as gametogenesis, which is followed by fertilization, with the union of male and female gametes originating a zygote. This zygote migrates and adheres to the bowel epithelium, where it develops into an oocyst. When oocyst ruptures, it releases sporozoites which go to the salivary gland and are released during mosquito feeding (Ghosh A, Edwards M J & Jacobs-Lorena M (2000) The journey of the malaria parasite into the mosquito: Hopes for the new century. Parasitol Today 16: 196-201).

Besides the great variety of parasite forms in the host and vector mosquito, a noticeable feature of the life cycle of several species of Plasmodium is its synchronization and periodicity. Such distinguished periodicity in formation of gametocytes, the sexual forms of parasite, have been observed since the beginning of last century, and all research done with several species of Plasmodium show the existence of a gametocyte production peak at night, every 24 hours, usually at the same time of mosquito feeding. In this way, the gametocytes circadian rhythm must be an important adaptation for maintenance of parasite sexual cycle in the vector mosquito (Garcia C R S, Markus R P & Madeira L (2001) Tertian and quartan fevers: temporal regulation in malarial infection. J Biol Rhythms 16: 436-443). Until now the signal responsible for inducing gametocytes formation in the vertebrate host bloodstream was not identified.

Regarding asexual forms, the high synchronization of Intraerythrocytic stages results in recurring fever attacks and shivers, always in periods of time multiple of 24 hours, coinciding with a practically simultaneously release of billion of merozoites in bloodstream. This is an important mechanism of evasion of host immune system which has arisen researchers' interest for decades.

In 2000, in a study conducted by our laboratory, Hotta et al. reported from in vitro, and in vivo trials with surgically (the pineal gland removal) and pharmacologically (using luzindole, an melatonin antagonist) pinealectomized mices, that melatonin synchronizes the maturation stages of P. chabaudi and P. falciparum. It was also evidenced that, in vitro, this hormone causes the release of Ca²⁺ from Plasmodium intracellular supplies. Effects of melatonin on parasite cycle are blocked by a phospholipase C inhibitor (U73122), suggesting a possible melatonin action mechanism by binding receptors coupled to G protein, causing the activation of phospholipase C and increase in intracellular Ca²⁺ levels via IP₃ (Hotta C T, Gazarini M, Beraldo F H, Varotti F P, Lopes C, Markus R P, Pozzan T & Garcia C R S (2000) Calcium-dependent modulation by melatonin of the circadian rhythm in malarial parasites. Nature Cell Biology 2: 466-468). Probably the circadian changes in the concentration of such hormone produced by the host represent a key-signal for maturation synchronized control of this parasite in vivo.

### Intracellular Signaling in Plasmodium

The complex life cycle of malaria parasites is characterized by successive stages of specialized development and each of them is essential for cycle continuance. Several micro-arrangement studies made for determining the P. falciparum genome expression pattern revealed that parasite Intraerythrocytic stages have specialized mechanisms for transcriptional regulation which result in a continuous cascade of gene expression with correlated functions (Bozdech Z, Llina M, Pulliam B L, Wong E D, Zhu J & DeRisi J L (2003) The Transcriptome of the Intraerythrocytic Developmental Cycle of Plasmodium falciparum. PLOS Biology 1: 1-16; Le Roch K G, Zhou Y, Blair P L, Grainger M, Moch J K, Haynes J D, De La Vega P, Holder A A, Batalov S, Carucci D J & Winzeler E A (2003) Discovery of gene function by expression profiling of the malaria parasite life cycle. Science 301: 1503-1508). Furthermore, it has been evidenced that some stages of Plasmodium life cycle are capable of responding to signs from the vertebrate host or vector-insect, so that its cell differentiation process be in synchrony with the environment at which the parasite lives (Hotta C T, Gazarini M, Beraldo F H, Varotti F P, Lopes C, Markus R P, Pozzan T & Garcia C R S (2000) Calcium-dependent modulation by melatonin of the circadian rhythm in malarial parasites. Nature Cell Biology 2: 466-468; Beraldo F H, Almeida F M, da Silva A M & Garcia C R S (2005) Cyclic AMP and calcium interplay as second messengers in melatonin-dependent regulation of Plasmodium falciparum cell cycle. J Cell Biol 170: 551-557; Garcia G E, Wirtz R A, Barr J R, Woolfitt & Rosenberg R (1998) Xanthurenic acid induces gametogenesis in Plasmodium, the malaria parasite. J Biol Chem 273 (20): 12003-12005).

Gametogenesis in mosquito vector bowel may be used as an example of the importance of studies on signaling paths involved in environment perception and parasite physiological response, activating a morphogenesis process which results in an advancement on parasite cell cycle towards the formation of mature gametes for fertilization. Xanthurenic acid, a molecule produced in the mosquito salivary gland from tryptophan metabolism, was identified as a factor derived from the vector insect capable of inducing male gamete exflagellation (Billker O, Lindo V, Panico M, Etienne A E, Paxton T, Dell A, Rogers M, Sinden R E & Morris H R (1998) Identification of xanthurenic acid as the putative inducer of malaria development in the mosquito. Nature 392: 289-292; Garcia G E, Wirtz R A, Barr J R, Woolfitt & Rosenberg R (1998) Xanthurenic acid induces gametogenesis in Plasmodium, the malaria parasite. J Biol Chem 273 (20): 12003-12005.; Hirai M, Yoshida S, Ishii A & Matsuoka H (2001) Characterization and identification of exflagellation-inducing factor in the salivary gland of Anopheles stephensi (Diptera: Culicidae). Biochem Biophys Res Comm 287: 859-864). Among the action mechanisms induced by XA are the membrane phospholipids hydrolysis during exflagellation, generating IP₃ and DAG, the release of calcium from parasite intracellular supplies and increase in intracellular levels of GMPc (Kawamoto F, Alejo-Blanco R, Fleck S L, Kawamoto Y & Sinden R E (1990) Possible roles of Ca²⁺ and cGMP as mediators of the exflagellation of Plasmodium berghei and Plasmodium falciparum. Mol Biochem Parasitol 42: 101-108.; Martin S K, Jett M & Scheneider I (1994) Correlation of phosphoinositide hydrolysis with exflagellation in the malaria microgametocyte. J Parasitol 80: 371-378. Billker O, Dechamps S, Tewari R, Wenig G, Franke-Fayard B & Brinkmann V (2004) Calcium and calcium-dependent protein kinase regulate gamete formation and mosquito transmission in a malaria parasite. Cell 117: 503-514). Besides, it has been also evidenced that as a result of the production of such second messengers occurs activation of effectoring enzymes such as guanylyl cyclase and CDPK4 calcium-dependent kinase (Muhia D K, Swales C A, Deng W, Kelly J M & Baker D A (2001) The gametocyte-activating factor xanthurenic acid stimulates an increase in membrane-associated guanylyl cyclase activity in the human malaria parasite Plasmodium falciparum. Mol Microbiol 42: 553-60; Billker O, Dechamps S, Tewari R, Wenig G, Franke-Fayard B & Brinkmann V (2004) Calcium and calcium-dependent protein kinase regulate gamete formation and mosquito transmission in a malaria parasite. Cell 117: 503-514). CDPK4 calcium-dependent protein kinase was identified as one of the calcium molecular targets which translate the XA signal into a regulation response of the cell cycle progression in male gametocyte.

Concerning parasite asexual cycle, our laboratory evidenced that melatonin, circadianly produced by vertebrate host pineal gland, synchronizes asexual stages of P. chabaudi and P. falciparum (Hotta C T, Gazarini M, Beraldo F H, Varotti F P, Lopes C, Markus R P, Pozzan T & Garcia C R S (2000) Calcium-dependent modulation by melatonin of the circadian rhythm in malarial parasites. Nature Cell Biology 2: 466-468.; Hotta C T, Markus R P & Garcia C R S (2003) Melatonin and N-acetyl-serotonin cross the red blood cell membrane and evoke calcium mobilization in malarial parasites. Braz J Med Biol Res 36:1583-7). Melatonin is a lipophilic molecule capable of crossing biological membranes, in a way that can interact with both extracellular and intracellular targets. Hotta C T, Markus R P & Garcia C R S (2003) Melatonin and N-acetyl-serotonin cross the red blood cell membrane and evoke calcium mobilization in malarial parasites. Braz J Med Biol Res 36:1583-7. Beraldo F H, Almeida F M, da Silva A M & Garcia C R S (2005) Cyclic AMP and calcium interplay as second messengers in melatonin-dependent regulation of Plasmodium falciparum cell cycle. J Cell Biol 170: 551-557, showed that melatonin is capable of causing mobilization of P. chabaudi and P. falciparum calcium intracellular supplies even in intact infected erythrocytes, indicating that it must be able to cross erythrocyte membranes and parasitophorous vacuole, and then activate parasite membrane receptors. Gazarini M L, Thomas A P, Pozzan T & Garcia C R S (2003) Calcium signaling in a low calcium environment: how the intracellular malaria parasite solves the problem. J Cell Biol 161: 103-110, evidenced that parasitophorous vacuole is a calcium-rich microenvironment, which is essential for creating conditions to calcium-mediated intracellular signaling.

Furthermore, it was shown that other products of tryptophan catabolism such as N-acetylserotonin, serotonin and tryptamine, are also capable of synchronizing P. falciparum cycle and mobilizing Ca²⁺ (Beraldo, F H & Garcia C R S (2005) Products of tryptophan catabolism induce a Ca²⁺ release and modulate the cell cycle of P. falciparum malaria parasites. J Pineal Res 39: 224-230). Among all intracellular targets of melatonin is a Ca²⁺-dependant thiol protease (Farias S L, Gazarini M L, Melo R L, Hirata I Y, Juliano M A, Juliano L & Garcia C R S (2005) Cysteine-protease activity elicited by Ca(2+) stimulus in Plasmodium. Mol Biochem Parasitol 141: 71-79). Besides the second Ca²⁺ messenger, Beraldo, F H & Garcia C R S (2005) Products of tryptophan catabolism induce a Ca²⁺ release and modulate the cell cycle of P. falciparum malaria parasites. J Pineal Res 39: 224-230). Beraldo F H, Almeida F M, da Silva A M & Garcia C R S (2005) Cyclic AMP and calcium interplay as second messengers in melatonin-dependent regulation of Plasmodium falciparum cell cycle. J Cell Biol 170: 551-557, showed that melatonin induces the increase of AMPc and PKA activation, an event that is preceded by an increase in intracellular Ca²⁺ once it is stopped by phospholipase C (U73122) inhibitor and by intracellular Ca²⁺ chelator BAPTA-AM. In fact, it was shown that AMPc generated in response to melatonin also induces release of Ca²⁺, which evidences a complex relation between the paths of these two second messengers in malaria parasites (Beraldo F H, Almeida F M, da Silva A M & Garcia C R S (2005) Cyclic AMP and calcium interplay as second messengers in melatonin-dependent regulation of Plasmodium falciparum cell cycle. J Cell Biol 170: 551-557). Thus, melatonin-activated signaling paths in Plasmodium indicates a G protein-coupled receptors-mediated signaling (GPCRs), once they imply activation of C phospholipase and adenylyl cyclase, producing Ca²⁺ second messengers and cAMP (Hotta C T, Gazarini M, Beraldo F H, Varotti F P, Lopes C, Markus R P, Pozzan T & Garcia C R S (2000) Calcium-dependent modulation by melatonin of the circadian rhythm in malarial parasites. Nature Cell Biology 2: 466-468.; Beraldo F H, Almeida F M, da Silva A M & Garcia C R S (2005) Cyclic AMP and calcium interplay as second messengers in melatonin-dependent regulation of Plasmodium falciparum cell cycle. J Cell Biol 170: 551-557).

Several studies have identified proteins which are involved in the intracellular signaling cascade of P. falciparum, such as adenylyl cyclase (AC), guanylyl cyclase (GC), PKA, PKG, RACK, Ca²⁺-ATPase, CDPKs, calmodulin and MAPKs (Aravind L, Iyer L M, Wellems T E & Miller L H (2003) Plasmodium biology: genomic gleanings. Cell 115: 771-785; Baker D A & Kelly J M (2004) Purine nucleotide cyclases in the malaria parasite. TRENDS in Parasitol 20: 227-232.; Madeira L, DeMarco R, Gazarini M L, Verjovski-Almeida S & Garcia C R S (2003) Human malaria parasites display a receptor for activated C kinase ortholog. Biochem Biophys Res Comm 306: 995-1001; Ward P, Equinet L, Packer J and Doerig C (2004) Protein kinases of the human malaria parasite Plasmodium falciparum: the kinome of a divergent eukaryote. BMC Genomics 5: 79; Khan S M, Franke-Fayard B, Mair G R, Lasonder E, Janse C J, Mann M & Waters A P (2005) Proteome analysis of separated male and female gametocytes reveals novel sex-specific Plasmodium biology. Cell 121: 675-687; Anamika, Srinivasan N & Krupa A (2005) A genomic perspective of protein kinases in Plasmodium falciparum. Proteins 58: 180-189). However, in spite of P. falciparum genome project conclusion (Gardner M J, Hall N, Fung E, White O, Berriman M, Hyman R W, Carlton J M, Pain A, Nelson K E, Bowman S, Paulsen I T, James K, Eisen J A, Rutherford K, Salzberg S L, Craig A, Kyes S, Chan M S, Nene V, Shallom S J, Suh B, Peterson J, Angiuoli S, Pertea M, Allen J, Selengut J, Haft D, Mather M W, Vaidya A B, Martin D M, Fairlamb A H, Fraunholz M J, Roos D S, Ralph S A, McFadden G I, Cummings L M, Subramanian G M, Mungall C, Venter J C, Carucci D J, Hoffman S L, Newbold C, Davis R W, Fraser C M & Barrell B (2002) Genome sequence of the human malaria parasite Plasmodium falciparum. Nature 419: 498-511), proteins which act at the beginning of signaling cascade, or the receptors of extracellular signs and proteins that make mediation between them and their effectors, still have not been identified.

Considering the importance of acknowledging extracellular signs from host/vector by malaria parasites in such a way that their own cell cycle be regulated according to the environment where they are located, identification of signaling paths proteins is essential for explaining biological mechanisms of such great relevance for the parasite-host relationship which may contribute to the production of antimalaricals.

### SUMMARIZED DESCRIPTION

The present invention relates to a pharmaceutical composition comprising one or more compounds that bind to serpentine receptors existent in parasites of genus Plasmodium besides pharmaceutically acceptable excipients. The invention also includes the drug screening method and method for treating malaria.

### DETAILED DESCRIPTION

Although serpentine receptors are well-known, membrane receptors for extracellular signs are still unknown in P. falciparum.

Serpentine receptors are proteins comprised of seven-transmembrane domains that act in the molecular recognition.

G protein-coupled receptors (GPCRs), generally called as serpentine or heptahelical receptors. Serpentine receptors are proteins comprised by seven-transmembrane domains that act in the molecular recognition, such receptors mediate physiological responses to several stimulus such as light, smells, pheromones, hormones, neurotransmitters, small peptides, proteins, lipids and ions (Hall R A, Premont R T & Lefkowitz R J (1999) Heptahelical receptor signaling: beyond the G-protein paradigm. J Cell Biol 145: 927-932).

According to the classical view, GPCRs couple with effectoring proteins such as adenylyl or guanylyl cyclases, A2 or C phospholipases and ionic channels, via heterotrimerics proteins which bind to guanine nucleotides (G proteins). However, it is now evident that many heptahelical receptors-mediated processes operate independently from G proteins (Hall R A, Premont R T & Lefkowitz R J (1999) Heptahelical receptor signaling: beyond the G-protein paradigm. J Cell Biol 145: 927-932.; Brzostowski J A & Kimmel A R (2001) Signaling at zero G: G-protein-independent functions for 7-TM-receptors. TRENDS Biochem Sci 26: 291-297.). GPCRs are the most expanded class of membrane receptors, with members in bacteria, fungus, plants and all metazoan organisms. Despite of its preserved structure comprised by seven-transmembrane domains (7-TM), GPCRs are highly divergent, with each family member sharing only 25% of identity at the amino acid level within the preserved transmembrane region, while a little similarity is shared among different families (Pierce K L, Premont R T & Lefkowitz R J (2002) Seven-transmembrane receptors. Nat Rev Mol Cell Biol 3:639-50.).

Thus, the present invention relates to a phamarceutical composition comprising one or more compounds that bind to serpentine receptors existent in parasites of genus Plasmodium besides pharmaceutically acceptable excipients. Serpentine receptors may belong to the family of: rodopsines (family A), secretines (family B) and metabotropic glutamate receptors (family C). In addition serpentine receptors may be G protein-dependent or independent.

Receptors may be present in the following species of genus Plasmodium: Plasmodium falciparum, Plasmodium chabaudi, Plasmodium yoelli, Plasmodium vivax, Plasmodium malariae, Plasmodium berghei.

Pharmaceutical composition may be used by oral, parenteral, rectal or topic route. If it is orally, tablets, pills, powders (gelatin capsules, communion wafer) or pellets, solutions, suspensions, emulsions, syrups and pharmaceutically acceptable elixirs may be used. For parenteral administration, aqueous or non aqueous solutions, suspensions or emulsions may be preferable. Rectal administration compositions are suppository or rectal capsules. For topic administration, for instance, creams, lotions, eye drops, mouthwashes, nasal drops or aerosols may be used. The invention also describes the screening method and method for treating malaria using serpentine receptors existent in parasites of genus Plasmodium.

Serpentine receptors may be classified as: rodopsines (family A), secretines (family B) and metabotropic glutamate receptors (family C). Serpentine receptors may be G protein-dependent or independent.

Compounds that bind to serpentine receptors may be: pheromones, hormones, neurotransmitters, small peptides, proteins, lipids and ions.

Receptors may be present in the following species of Plasmodium genus: Plasmodium falciparum, Plasmodium chabaudi, Plasmodium yoelli, Plasmodium vivax, Plasmodium malariae, Plasmodium berghei.

Screening method employs gene transfection of the serpentine receptors in mammalian cells. After expression of such genes in a heterologous system, the change in calcium concentration of the cell shall be measured by adding several potential ligands for serpentine receptors. Following is an example for better explaining the scope of invention without serving as a basis for restrictive effect of invention.

### EXAMPLE 1

### Drug Screening Method

### Ca²⁺ Functional Essays

Cells were washed three times with 200 μL of DMEM without serum and marked with Fluo-4 AM (5 μM) in DMEM also without serum for one hour at 37° C. After marking, cells were washed three times with 200 μL of HBSS buffer (5.4 mM KCl, 0.3 mM Na2HPO4, 0.4 mM KH2PO4, 4.2 mM NaHCO3, 0.5 mM MgCl2, 0.6 mM MgSO4, 137 mM NaCl, 5.6 mM glucose) containing 2 mM of CaCl₂. A confocal microscopy was used for imaging acquisition (laser scanning microscopy LSM 510-Carl Zeiss) using LSM 510 software, version 2.5. The objective used shall be of 40× (oil immersion). Samples were excited at 488 nm with argon laser and fluorescence issued was collected with a 505-530 nm bandpass filter. Essays consisted of adding drugs which response were intended to be tested.

### 2-[¹²⁵I]-Iodomelatonin Binding Essay

COS-7 cells transfected with candidates to serpentine receptor were centrifuged at 3000 g during 5 minutes and stored at −70 ° C. until use. Cells were washed two times in a binding buffer (10 mM Tris-HCl, 1 mM EDTA pH 7.5). 1×10⁶ cells were incubated at 37° C. for 2 hours with 100 pM 2-[¹²⁵I]-iodomelatonin in 200 μL of binding buffer with or without melatonin 10⁻⁶ M for detecting specific binding. Reaction was stopped by cooling samples with ice rapidly, followed by addition of 0.1% of sheep γ-globulin and 1 mL of PEG 8000 24% dissolved in a cold binding buffer. Fraction bound to 2-[¹²⁵I]-iodomelatonin was recovered by centrifugation during 30 minutes at 1800 g at 4° C. Supernatant was discarded and precipitate was resuspended at 12% of PEG 8000 and 0.05% of sheep γ-globulin. Precipitate was recovered through a new centrifugation and dried at room temperature (Conway et al. 1997). Radioactivity was detected by scintillometer (Tri-Carb 2100 TR Packard).

### Gene Codon-Optimization

The complete ORF sequence of putative receptor was commercially codon-optimized (DNA 2.0). In order to increase the expression in mammalian cells, a Kozak consensus sequence (GCCGCC) was added at extremity 5′ of construction and FLAG epitope shall be added at extremity 3′ for monitoring expression in the heterologous system.

1. A pharmaceutical composition comprising one or more serpentine receptors-binding compounds existent in parasites of genus Plasmodium besides pharmaceutically acceptable excipients.

2. A pharmaceutical composition according to claim 1, wherein receptors may belong to the family of rodopsines (family A), secretines (family B) and metabotropic glutamate receptors (family C).

3. A pharmaceutical composition according to claim 1, wherein serpentine receptors may be G protein-dependent or independent.

4. A pharmaceutical composition according to claim 1, wherein compounds may be pheromones, hormones, neurotransmitters, small peptides, proteins, lipids and ions.

5. A pharmaceutical composition according to claim 1, wherein receptor may be present in the following species: Plasmodium falciparum, Plasmodium chabaudi, Plasmodium yoelli, Plasmodium vivax, Plasmodium malariae, Plasmodium berghei.

6. A drug screening method, wherein employs serpentine receptors existent in parasite of genus Plasmodium.

7. A drug screening method according to claim 6, wherein it employs the following technique: the calcium increase or AMPC is measured after screening employing potential ligands in cells transfected with genes candidates to serpentine receptors.

8. A method for treating malaria, comprising one or more serpentine receptors-binding compounds existent in parasites of genus Plasmodium.

9. A method for treating malaria according to claim 8, wherein receptors may belong to the family of: rodopsines (family A), secretines (family B) and metabotropic glutamate receptors (family C).

10. A method for treating malaria according to claim 8, wherein receptors may be G protein-dependent or independent.

11. A method for treating malaria according to claim 8, wherein compounds may be pheromones, hormones, neurotransmitters, small peptides, proteins, lipids and ions.

12. A method for treating malaria according to claim 8, wherein receptors may be present in the following species of genus Plasmodium: Plasmodium falciparum, Plasmodium chabaudi, Plasmodium yoeffi, Plasmodium vivax, Plasmodium malariae, Plasmodium berghei.
